(19) United States
(12) Patent Application Publication
(10) Pub. No.: US 2004/0230156 A1
(43) Pub. Date: Nov. 18, 2004

(54) Methods and devices for in-situ crosslinking of vascular tissue

(76) Inventors: Stefan Georg Schreck, Vista, CA (US); Erich Horst Wolf, Vista, CA (US); Ross Tsukashima, San Diego, CA (US)

Correspondence Address: STEFAN SCHRECK, ELYTRA MEDICAL LLC, SUITE G, 13670 DANIELSON STREET, POWAY, CA, 92064, US

(21) Appl. No.: 10/606,952

(22) Filed: Jun. 27, 2003

Related U.S. Application Data
(60) Provisional application No. 60/462,668, filed on Apr. 11, 2003.
(60) Provisional application No. 60/447,375, filed on Feb. 13, 2003.

(51) Int. Cl. A61M 29/00

(52) U.S. Cl. 604/96.01; 604/20

(57) ABSTRACT

A method for treating vulnerable plaque by crosslinking the fibrous cap, or collagenous extra cellular matrix layer, on the inner wall or vascular intima of the vessel. The method includes providing a vascular catheter, delivering a crosslinking agent to the extra cellular matrix layer with the vascular catheter, and irradiating the extra cellular matrix layer and crosslinking agent with light energy emitted from the vascular catheter. The crosslinking agent may be a saccharide, Riboflavin or Riboflavin-5-phosphate, or a photooxidizer. The irradiation energy is emitted from one or more LEDs mounted on the catheter. Catheter balloons are used to clear the optical path of blood so that short wavelength light can be used. The balloons may also be used to facilitate delivery of the crosslinking agent to the extra cellular matrix layer. A perfusion lumen ensures continuous blood flow during the procedure.

### RELATED APPLICATION

The present application claims priority under 35 U.S.C §119(e) to provisional application No. 60/462668, filed on Apr. 11, 2003 under the title “Method and Device for In-Situ Cross-linking of Tissue” and to provisional application No. 60/447375, filed on Feb. 15, 2003 under the title “Catheter for Delivery and Activation of a Photosensitizer”.

### FIELD OF INVENTION

The present invention relates to a method and catheter-based device for the in-situ crosslinking of vascular tissue.

### BACKGROUND OF THE INVENTION

Cardiovascular disease is one of the leading causes of death in the developed countries. It is estimated that more than one million people in the United States suffer from a sudden cardiac event each year. For a long time, coronary artery occlusions have been believed to be the main cause of sudden cardiac events. The occlusion of coronary arteries reduces the blood flow to the myocardium. In cases of severe occlusion and high cardiac workload, myocardial muscle cells do not receive sufficient oxygen and die. Clinical interventions for occlusion of coronary artery have focused on removing the blockage in the arteries. This is accomplished by expanding the artery with a balloon (balloon angioplasty), placement of stents in the lesion to keep the artery patent, or coronary bypass surgery with a vein graft. Despite the effectiveness of these procedures in treating stenotic lesions, patients still suffer from sudden cardiac events even in the absence of stenotic lesions.

Over the past several years, the attention of research into sudden cardiac events has shifted to vulnerable plaque, a rupture-prone plaque in the walls of coronary arteries. Vulnerable plaque is characterized by a large lipid pool in the plaque, a thin fibrous cap separating the plaque from the blood stream, and an inflammatory process within the plaque. Macrophages that infiltrate the fibrous cap break down the collagen structure of the cap by enzymatic degradation. The cap becomes too weak to withstand high hemodynamic loads and ultimately ruptures exposing the highly thrombogenic content of the plaque to the bloodstream. Thrombi form rapidly and can cause partial or complete occlusion of the blood vessel. It is believed that vulnerable plaque may be responsible for as many as 60-80% of all sudden cardiac events.

Vulnerable plaque is not only found in the coronary artery but in the whole arterial system of “vulnerable patients.” Rupture of vulnerable plaque in the ascending aorta is believed to be a main cause of stroke as the thrombus released from the plaque travels through the carotid arteries into the brain. Vulnerable plaque has also been found in the carotid arteries themselves. Approximately 600,000 Americans suffer a stroke each year. One-third die within one year and another third have severe disability.

Vulnerable plaque is difficult to research because current imaging systems are not capable of detecting the plaque in the vessel wall. Therefore, the investigation into vulnerable plaque has been limited to the biophysical and biochemical analysis of cadavers and retrospective studies of patients who have suffered a sudden cardiac event. Only some of the key findings of the ongoing research into vulnerable plaque are highlighted here.

Lipid-rich plaques seem to exist throughout the coronary and vascular systems of high-risk patients with “hot spots” of increased inflammatory activities. The size of vulnerable plaque in coronary arteries is typically less than 1 cm in length and covers approximately one-quarter to one-half of the circumference of the blood vessel. The fibrous cap has a thickness of less than 100 μm. A slight stenosis of the vessel may be present in some cases. Rupture of plaque seems to increase during periods of elevated physical activity or mental stress. Retrospective studies have identified several patient specific risk factors associated with sudden cardiac death. They include hypercoagulable blood, presence of serum markers of atherosclerosis and inflammation, and pre-existing atherosclerosis-related myocardial damage.

Various drugs are being studied for inhibiting the build up of lipids in the plaque and reducing the inflammatory process in the plaque. Drugs such as statins, anti-inflammatory agents, and angiotensin-converting enzyme (ACE) inhibitors have shown promising early results in reducing the risk of plaque rupture.

Several technologies have been proposed for the detection of vulnerable plaque. The inflammatory process in the vulnerable plaque causes a local rise in the temperature of the vessel wall that may be detected by temperature sensors. For instance, U.S. Pat. No. 6,514,214 to SciMed Life Systems, Inc. of Maple Grove, Minn. discloses catheter-based devices and methods for detecting vulnerable plaque within a blood vessel including at least one temperature sensor disposed proximate to the distal end of the catheter. Other researchers are investigating the possibility of using measurable changes in the systolic-diastolic extension of the vessel wall to detect the soft lipid-rich pool in vulnerable plaque. Others are utilizing direct imaging systems to visualize structures in the vessel wall. It is anticipated that some of these technologies will become clinically available within the next few years.

These detection advances provide a need and opportunity for local treatment modalities. Drug-eluding stents have been proposed for treatment of vulnerable plaque. Although vessel occlusion is not critical in vulnerable plaque, stents may support the thin fibrous cap while applying time-released drugs to suppress the inflammatory reaction. One major shortcoming of stenting is the need for several stents in cases of multiple lesions and the high cost of drug-eluding stents.

MedVenture Technology Corp. of Louisville, Ky. discloses two ways to treat vulnerable plaque in U.S. Pat. Nos. 6,419,659 and 6,475,210. Both patents disclose a catheter arrangement for the treatment of a lipid pool at a site of vulnerable plaque within an artery. The '659 catheter includes a needle that penetrate the fibrous cap and suctions the lipid material from its pool beneath the fibrous cap and adjacent to the artery wall. A treating agent may be injected within the fibrous cap to facilitate removal of the lipid therefrom or to promote healing of the artery wall once the aspiration catheter and steerable needle have been removed therefrom. The fluid introduced into the lipid pool may be a pharmaceutical agent to render the lipid non-thrombogenic or to facilitate its solidification. The '210 catheter has a distal end with an annular array of energy emitters arranged for the transmission of energy through the wall of a catheter sheath. The energy emitters communicate with an energy source at the proximal end of the catheter through a cable or optical fiber. The energy may be emitted in the microwave range, the ultrasound range, the infrared range, the ultraviolet range, or emitted as a tunable laser light so as to alter the lipid pool either through shrinking, congealing, or other effects.

Despite the aforementioned efforts, there remains a need for effective methods and devices for treatment of vulnerable plaque.

### SUMMARY OF THE INVENTION

The inventors have identified an alternative method for local treatment of vulnerable plaque that targets the thin fibrous cap. Research indicates that the fibrous cap is eroded by enzymatic degradation of the protein in the cap that ultimately causes the plaque to rupture. It is postulated that rupture of the plaque could be prohibited or at least significantly delayed by crosslinking the collagen in the fibrous cap to prevent enzymatic degradation.

The present invention involves a catheter for the delivery of a crosslinking agent and irradiation of the vascular tissue. The catheter contains high-intensity Light Emitting Diodes (LEDs) that are directly mounted on the tip of the catheter, a balloon to stabilize the catheter in target vessel, a delivery lumen to deliver the agent to the target site, and means for perfusion of the bodily vessel during the procedure.

In accordance with one aspect of the invention, a method for crosslinking an extra cellular matrix layer in the vascular system of the body includes the steps of:

providing a vascular catheter;

delivering a crosslinking agent to the extra cellular matrix layer with the vascular catheter; and

irradiating the extra cellular matrix layer and crosslinking agent with light energy emitted from the vascular catheter.

Desirably, the vascular catheter includes one or more light emitting diodes mounted thereon which provide the energy for the step of irradiating.

The crosslinking agent may contain Riboflavin or Riboflavin-5-phosphate. The wavelength of the irradiation energy is desirably between about 200 and 500 nm, and more preferably 220-225 nm, 266 nm, 371 nm, 444 nm, or 475 nm, which are the absorption maxima of Riboflavin.

Alternatively, the crosslinking agent contains a saccharide or a phosphate derivative thereof. The saccharide may be selected from the group consisting of:

glucose or a phosphate derivative thereof;

ribose or a phosphate derivative thereof; and

fructose or a phosphate derivative thereof.

In that case, the wavelength of the irradiation energy is desirably between about 150 and 400 nm.

Alternatively, the crosslinking agent contains a photooxidizer that generate crosslinks directly through the generation of oxygen radicals. Suitable photooxidizers include but are not limited to Aminolevulinic Acid, Psoralen, and 8-Methoxypsoralen, and 1,8-Naphthyridine. The wavelength of irradiation is tuned to the respective absorption wavelengths of the photooxidizers.

The crosslinking agent may further contain hydrogen peroxide, traces of metals or a photosensitizer that generates oxygen radicals when irradiated. The purpose of the oxygen radicals is to accelerate the crosslinking kinetics.

The present invention also provides a vascular catheter for delivering light energy to a blood vessel wall and crosslinking an extra cellular matrix layer thereon. The catheter includes a light-emitting diode (LED) on the distal end of the catheter, a transparent balloon mounted over the LED; and a lumen opening distal and proximal to the balloon. The lumen may be of sufficient size for blood perfusion therethrough, or may be suitable for passage of a guidewire.

The balloon may be mounted eccentrically onto the balloon to create a void between the catheter and the body vessel for perfusion of the vessel distal to the catheter. The balloon may also form a cavity between the outer surface of the balloon and the vessel wall for holding a therapeutic agent, such as with a dog-bone shape. A photosensitive agent may be contained in the balloon.

In one embodiment, there are at least two balloons mounted parallel to each other, and wherein the inflation of the balloons creates a void between the balloons, the catheter, and the vessel wall for perfusion of the vessel distal to the catheter. For instance, there are just two balloons mounted on opposite sides of the catheter and one is larger than the other and covers the LED. Alternatively, there are four balloons mounted along longitudinal quadrants of the catheter and connected to at least two inflation lumens. Four arrays of axially spaced LEDs are then mounted along the catheter and beneath the respective balloons.

### BRIEF DESCRIPTION OF THE DRAWINGS

FIGS. 1A and 1B are longitudinal and transverse sections, respectively, of a single-array illumination catheter of the present invention.

FIGS. 2A and 2B are longitudinal and transverse sections, respectively, of a multi-array illumination catheter of the present invention, and FIG. 2C is an alternative transverse section of a similar catheter.

FIGS. 3A and 3B are longitudinal and transverse sections, respectively, of a therapeutic catheter of the present invention with a central perfusion lumen and a balloon covering the LEDs, and FIGS. 3C and 3D are longitudinal and transverse sections, respectively, of the same catheter with the balloon inflated against the inner lumen of a surrounding vessel.

FIGS. 4A and 4B are longitudinal and transverse sections of a therapeutic catheter of the present invention with an eccentric balloon for partial treatment of the vessel wall, and FIGS. 4C and 4D are longitudinal and transverse sections, respectively, of the same catheter with the balloons inflated against the inner lumen of a surrounding vessel creating channels for perfusion.

FIGS. 5A and 5B are longitudinal and transverse sections of a therapeutic catheter of the present invention with a 4-compartment balloon for step-wise treatment of the vessel wall, and FIGS. 5C and 5D are longitudinal and transverse sections, respectively, of the same catheter with two opposing balloon compartments inflated against the inner lumen of a surrounding vessel creating channels for perfusion. FIG. 5E show a transverse section of the same balloon with the two other balloon compartments inflated.

FIGS. 6A-6C show longitudinal sections of a therapeutic catheter of the present invention having a dog-bone shaped balloon in several stages of deployment.

FIGS. 7A-7C show longitudinal sections of a further therapeutic catheter of the present invention having a non-uniformly inflating balloon in several stages of deployment.

FIGS. 8A-8C show longitudinal sections of yet another therapeutic catheter of the present invention having dual balloons, one permeable to a cross-linking agent, and in several stages of deployment.

### DESCRIPTION OF THE PREFERRED EMBODIMENTS

The present invention involves methods of treating vulnerable plaque by crosslinking the fibrous cap, or collagenous extra cellular matrix layer, on the inner wall or vascular intima of the vessel. The techniques described herein may produce some change in the underlying lipid pool, but the primary intent is protecting or strengthening the fibrous cap to help prevent future rupture thereof. The methods of the present invention are believed superior to those that treat the lipid pool because an area of the extra cellular matrix layer larger than the underlying lipid pool can be treated. This helps protect against ruptures of later forming vulnerable plaque deposits closely adjacent to the first.

Crosslinking of tissue in-situ requires that the crosslinking reaction produces biocompatible, non cytotoxic reaction products and does not damage collateral tissue. A group of biocompatible crosslinking agents have been identified that exist naturally in the body. These agents contain carbon sugar groups and are involved in the formation of advanced glycation endproducts. These agents can be absorbed by the body without harm. The natural reaction kinetics of the agents is slow but can be accelerated by appropriate means. For example, in diabetic patients elevated concentrations of glucose are found in the blood. High concentrations of glucose are known to crosslink the collagen in the wall of blood vessels. Besides increasing the concentration of the agent, the crosslinking reaction can further be accelerated in the presence of oxygen radicals. There are many strategies to generate oxygen radicals. For example hydrogen peroxide forms oxygen radicals when irradiated with UV light or when reacting with iron or copper. Alternatively, oxygen radicals can be generated by adding a photosensitizer to the crosslinking agent and irradiating the photosensitizer with light of the appropriate wavelength. Alternatively, the collagen in the tissue can be irradiated with UV light to release oxygen radicals. Certain crosslinking agents generate oxygen radicals themselves. For example, riboflavin is a photosensitizer that contains carbon sugars. When irradiated, it generates oxygen radicals that facilitate the crosslinking of its carbon sugar groups with proteins.

The following crosslinking agents/methods are being considered:

1. Riboflavin (Ribose attached to a flavin moiety). Riboflavin-5-phosphate (Vitamin B-2):

a. Activated by irradiation with light in the UV/blue spectrum preferably at wavelengths of between about 200 nm and 500 nm, and more preferably at about 220-225 nm, 266 nm, 371 nm, 444 nm, or 475 nm, which are the absorption maxima of Riboflavin.

2. A mono-saccharide (a simple sugar that cannot be hydrolysed to smaller units). The empirical formula of simple sugars is (CH₂O)n and range in size from trioses (n=3) to heptoses (n=7). For example, Glucose (n=6) and Ribose (n=5) or their phosphate derivatives, preferably Ribose-5-phosphate. Fructose is a polysaccharide that can also be used. The natural reaction kinetics of these sugars is slow but can be accelerated by combination with oxygen radicals by the following means:

a. Activated by irradiation of the tissue containing the agent with UV light at wavelengths of between about 150 nm and 400 nm. Collagen irradiated with UV light releases free radicals that accelerate the crosslinking reaction of glucose. Trace metals such as Iron (Fe) or Copper (Cu) may be added as catalysts (e.g. CuSO₄) to further accelerate the crosslinking reaction.

b. Activated by a photosensitizer that generates free radicals when irradiated. Suitable photosensitizers include but are not limited to Aminolevulinic Acid, Psoralen, and 8-methoxypsoralen. Trace metals such as Iron (Fe) or Copper (Cu) may be added as catalysts (e.g. CuSO₄) to further accelerate the crosslinking reaction.

c. Activated by hydrogen peroxide irradiated with UV light at wavelengths of between about 150 nm and 400 nm. Trace metals such as Iron (Fe) or Copper (Cu) may be added as catalysts (e.g. CuSO₄) to further accelerate the crosslinking reaction.

3. Photooxidizer that generate crosslinkings directly through the generation of oxygen radicals. Suitable photooxiders include but are not limited to Aminolevulinic Acid, Psoralen, and 8-methoxypsoralen, 1,8-Naphthyridine. The photooxiders are activated by irradiation with light containing the absorption wavelength of the respective photooxidizer agent.

The crosslinking agent can be delivered in a pH-buffered solution (e.g. phosphate buffer) of pH 7.4 to minimize collateral damage to living tissue. The concentration of the agents may be in the range from 0.01% to 20% preferably between 0.1% and 1.0%. In some cases it may be advantages to alter the pH of the solution in order to optimize the reaction kinetics.

The crosslinking agent is desirably delivered into the body with a catheter. A balloon on the distal end of the catheter stabilizes the catheter in the target vessel. A light source mounted at the distal end of the catheter irradiates the vessel wall to be crosslinked.

Current technologies for photodynamic therapies consider irradiation of the target site with a laser light. The laser light is channeled into the catheter and transmitted to the distal end of the catheter through an optical fiber. An optical deflector at the distal end of the catheter redirects the light beam to the vessel wall. The shortcoming of the technology is the need of a high-power laser and the non-uniformity of the light emission at the catheter tip. Furthermore, the optical fiber occupies significant space in the catheter and increases the stiffness of the catheter. The reduced space restricts the cross-sectional area needed for perfusion lumens, for example.

In contrast, the present invention includes the use of high-intensity light emitting diodes (LEDs) to irradiate the target site. The LEDs are directly mounted on the tip of the catheter eliminating the need for an optical cable in the catheter. Furthermore, use of arrays of LEDs or deposition of the LED(s) directly onto the catheter shaft will create a more uniform irradiation pattern for controlled activation of the photo-sensitive agent. Additionally, the LEDs of the present invention operate at relatively short wavelengths. For example, the wavelength of the light energy emitted by the LEDs of the present invention should be between about 150 and 500 nm, more preferably between about 250 nm and 480 nm, and in some cases between about 320 nm and 400 nm. As detailed above, the particular range is desirably tuned to a particular crosslinking agent or oxygen radical generator.

It should be noted that such short wavelength light energy has poor propagation characteristics through blood, and therefore a second challenge in irradiating the vessel wall is the need for a clear optical path from the LED to the vessel wall. One way in which this can be achieved is by inflating a transparent balloon with clear fluid to displace the blood in the vessel. The balloon can also be used to deliver the agent to the vessel wall. This can be accomplished in three ways: (1) the balloon is made from hydrophylic material such as a hydrogel, in which the agent is stored, (2) the balloon is made from permeable material and the inflation fluid contains the agent, and (3) the balloon(s) create a space or cavity between the balloon and the catheter to retain the agent during the procedure. Soft balloon material such as hydrogels and elastomers are considered desirable to minimize the inflation pressure. This is important when treating vulnerable plaque and other unstable sections of the vessel, where high local stresses may cause damage to the tissue. These techniques are possible using the devices described herein.

A third challenge is the obstruction of blood flow by the balloon during the procedure. Delivery and activation of the agent may take 10 to 60 minutes. It is critical to maintain perfusion of the blood vessel to avoid damage to the tissue distal to the catheter, especially in the coronary vessels. By placing the LEDs on the outer surface of the catheter, the central portion of the catheter can be used to house a perfusion lumen. Coincidentally, the blood passing through the central perfusion lumen can be used to cool the LEDs and avoid unwanted heating of the vessel walls. Alternatively, an eccentric balloon or an array of balloons that only partially occlude the vessel lumen may be used.

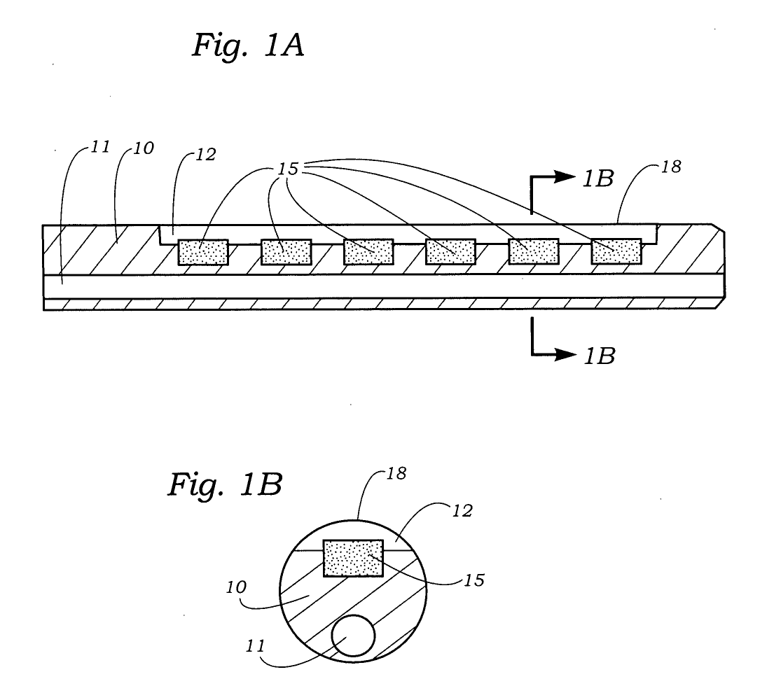

FIGS. 1A and 1B show a first embodiment of an illumination/irradiation catheter 10. A transverse section of the catheter is shown in FIG. 1B, and a longitudinal section is shown in FIG. 1A. The distal end of the catheter 10 has an elongated recess 12, in which the LEDs 15 are placed. A thin transparent cover 18 seals the LEDs against the blood flow. A central lumen 11 provides a passage for a guide wire.

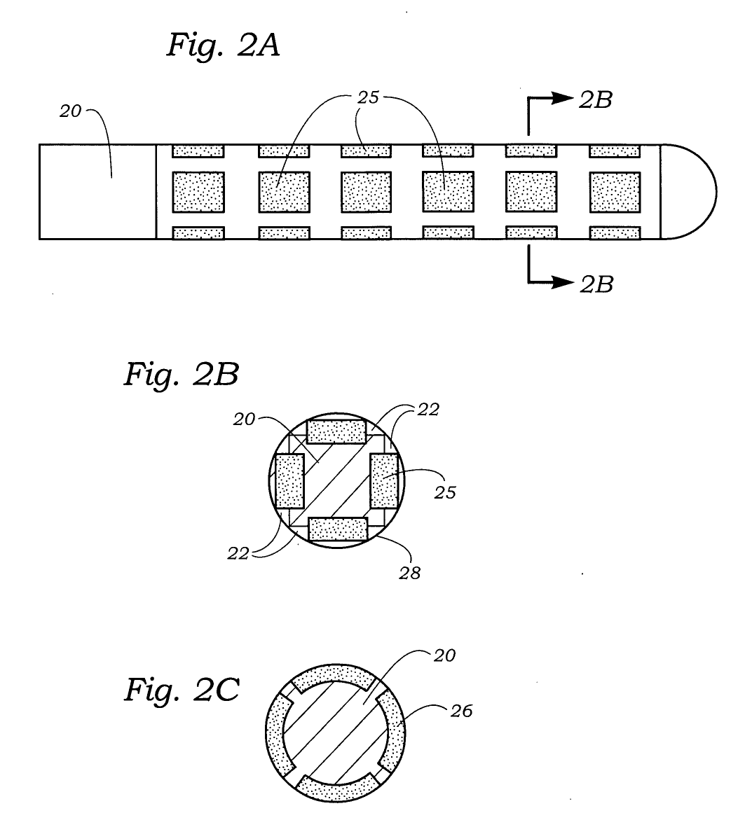

FIGS. 2A and 2B show an alternative configuration of an illumination/irradiation catheter 20. The distal tip of the catheter 20 is shown with four circumferentially distributed arrays of axially spaced LEDs 25 providing 360° illumination and/or irradiation of the vessel. The LEDs 25 are placed in four separate recesses 22 and are sealed with a cover 28 against the blood. The LED layers can also be deposited directly onto the shaft of the catheter, as seen in the alternative transverse section of FIG. 2C. By depositing the LED layers 26 directly onto the catheter 20, the LEDs conform to the surface of the catheter, thus increasing the overall surface area of the LEDs and creating a more uniform illumination pattern.

FIGS. 1 and 2 only show a few representative configurations of LEDs. It will be apparent to the skilled reader that the LEDs could be arranged in many different configurations depending on their application. For example, directly deposited LEDs may take the shape of rings, strips, or helices.

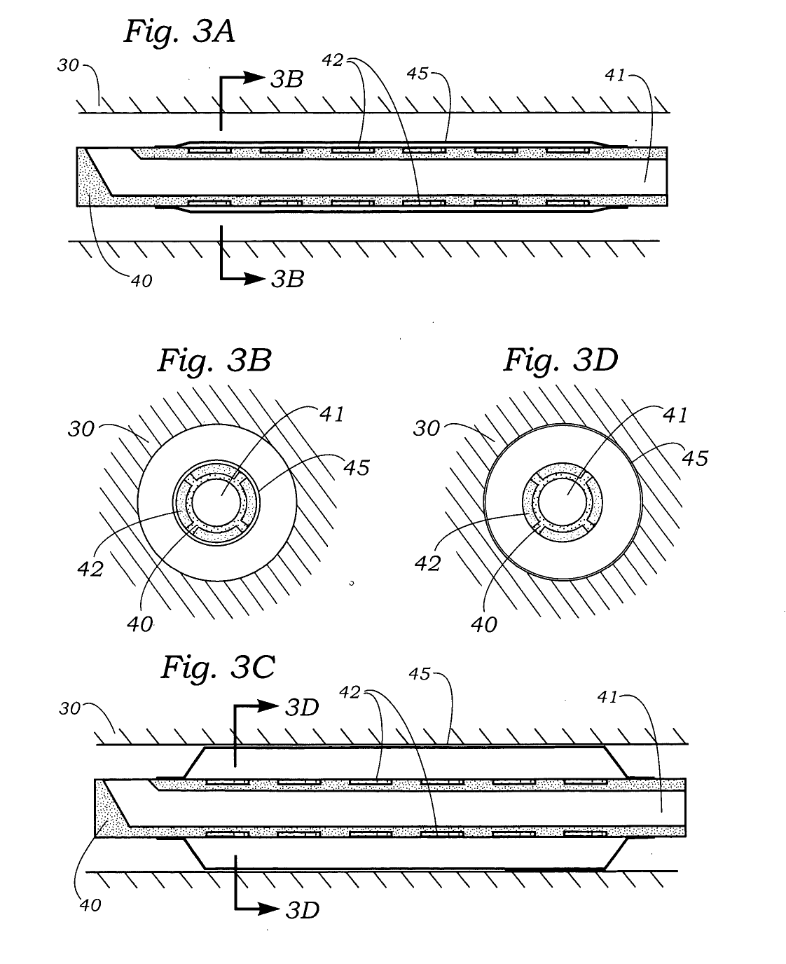

FIGS. 3A-3D show a therapy catheter 40 for the delivery and activation of a photo-sensitive agent. The catheter 40 has a central lumen 41 for perfusion, four arrays of LEDs 42 that are deposited onto the outer surface of the catheter 40, and a balloon 45. In FIGS. 3A and 3B a longitudinal and a transverse section, respectively, of a vessel 30 is shown with the tip of the catheter 40 centrally located therewithin. In FIGS. 3C and 3D the catheter is shown with the balloon 45 inflated and in contact with the vessel wall 30. The inflation fluid desirably contains the agent, which diffuses through the permeable balloon 45 into the vessel wall 30. When energized, the LEDs 42 irradiate the vessel wall and activate the agent. As can be seen in FIG. 3C, the perfusion lumen 41 allows blood to pass from the proximal end of the catheter tip to the distal end. The perfusion lumen 41 can also be utilized as a guide wire lumen.

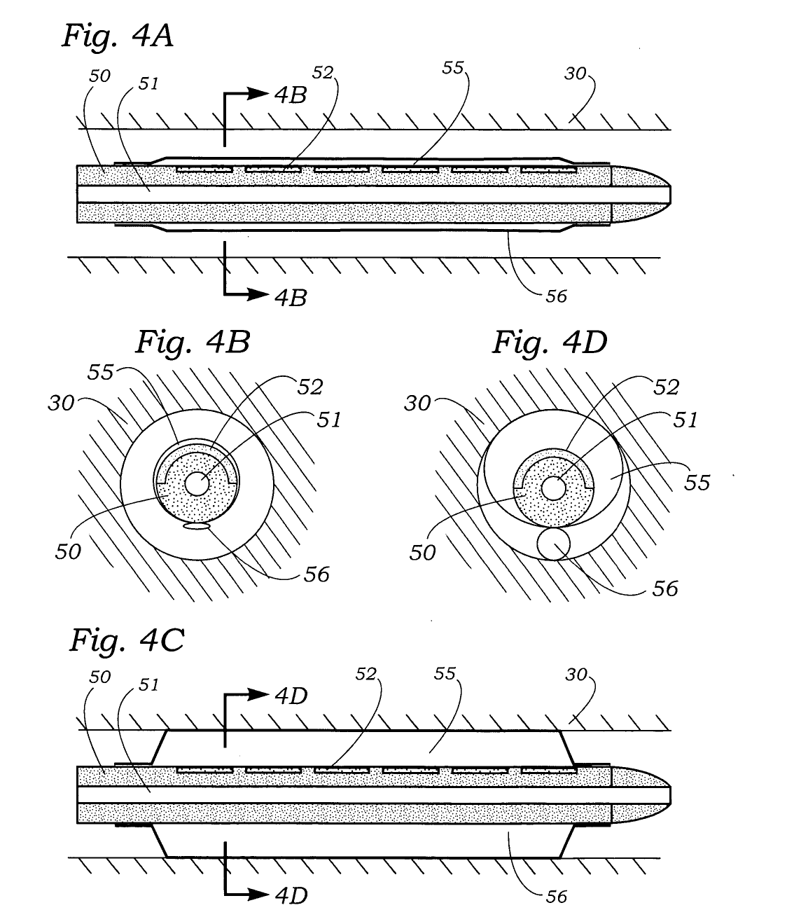

FIGS. 4A-4D show an alternative embodiment of a therapy catheter 50 designed to treat 180° of the vessel wall at a time. FIG. 4A is a longitudinal section of the catheter 50 illustrating the axial distribution of an array of LEDs 52. The transverse section of FIG. 4B shows the tip of the catheter 50 containing a central guide wire lumen 51. The arrays of LEDs 52 extend along one-half of the circumference of the catheter 50. Two eccentric balloons 55 and 56 are mounted on the balloon. The balloon 55 encloses the catheter 50 and the LEDs 52. The balloon 56 is mounted external to the catheter and opposite to the array of LEDs 52.

In FIGS. 4C and 4D the catheter 50 is shown with the balloons 55 and 56 fully inflated. The larger balloon 55 provides a clear light path for the LEDs to the vessel wall and desirably contains (e.g., is filled by) the agent. The balloon 56 acts as a stabilizing balloon centering the catheter 50 in the middle of the vessel. Two passages are created by the balloons 55 and 56 and the vessel wall 30 for perfusion of blood. To treat the whole vessel wall of a target vessel, first the irradiation is applied to one-half of the vessel wall. Then, the balloons 55 and 56 are deflated, the catheter 50 is rotated 180 degrees, the balloons 55 and 56 are re-inflated, and the second half of the vessel wall is irradiated.

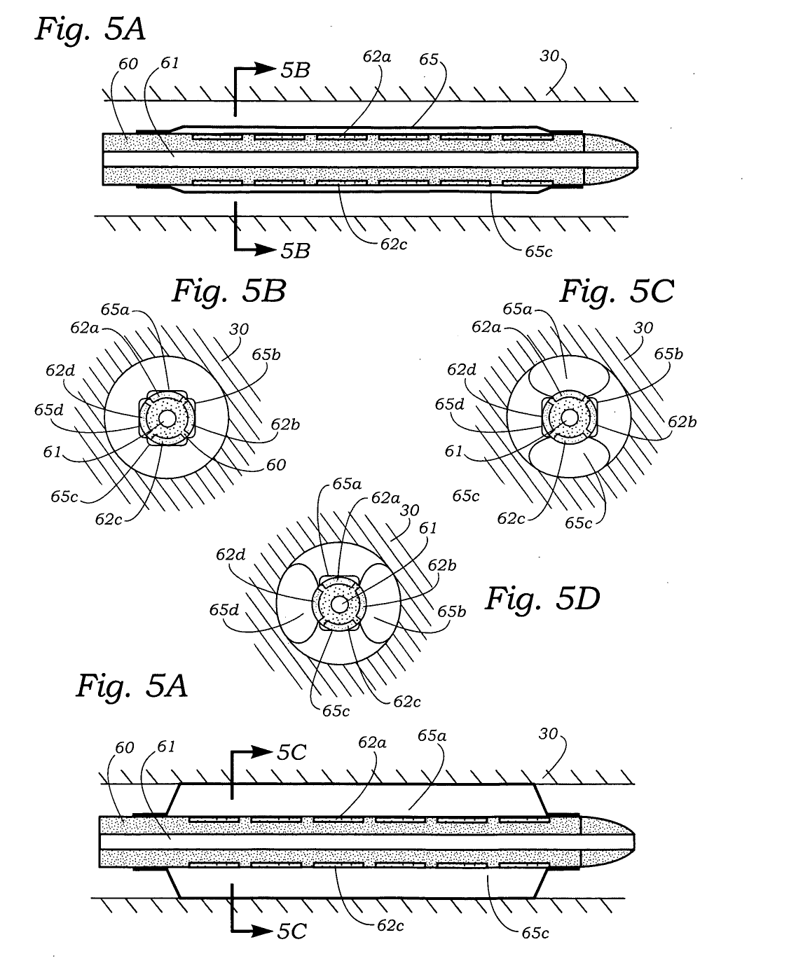

FIGS. 5A-5E show another embodiment of a therapy catheter 60 of the present invention containing a central guide wire lumen 61, four circumferentially distributed arrays of axially spaced LEDs 62a-62d, and four balloons 65a-65d. FIGS. 5D-5E illustrate two deployment configurations in transverse section.

In FIGS. 5C-5D opposed balloons 65a and 65c at six and twelve o'clock are inflated, and the other two balloons 65b and 65d at three and nine o'clock remain deflated. The arrays of LEDs 62a and 62c at three and nine o'clock are activated to irradiate opposing sections of the vessel wall.

In FIG. 5E opposed balloons 65a and 65c are deflated, and the balloons 65b and 65d are inflated. The LEDs 62b and 65d at three and nine o'clock are activated to irradiate the remaining sections of the vessel wall.

The balloons 65a-65d are desirably made of a highly elastic material, such as a hydrogel, that can by directly deposited onto the surface of the catheter during the manufacturing process. The surface of the balloon may be coated with Delrin or polyethylene or any other non-sticky material along the central section of the catheter tip that contains the LEDs to avoid adhesion of the balloon to the catheter when inflated. On the other hand, polyimide or polyethylene-terephthalate may be used to adhere the ends of the balloon to the catheter.

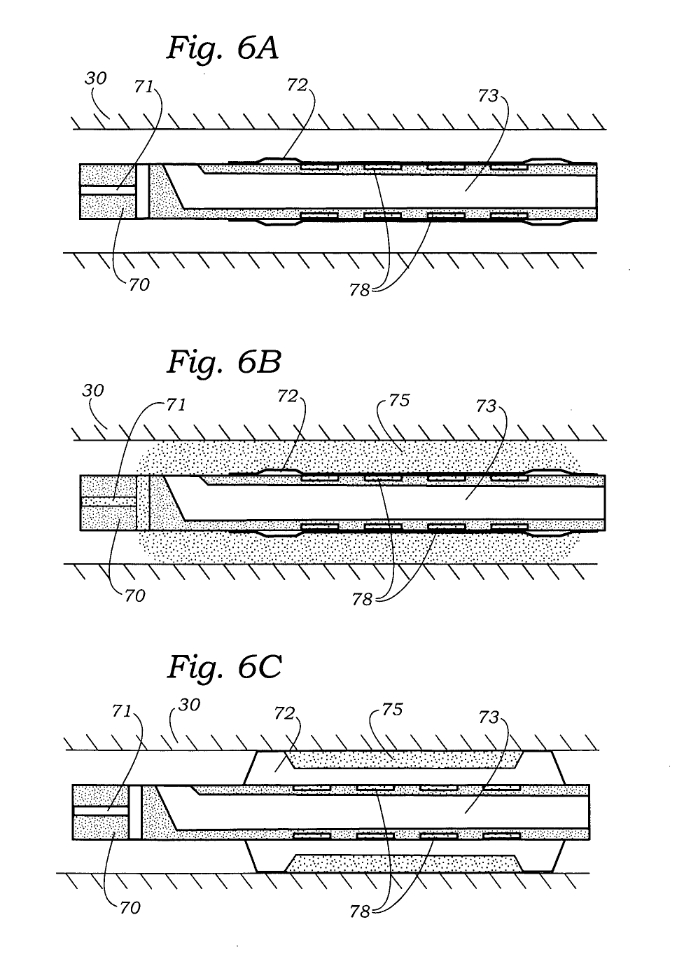

FIGS. 6A-C show another embodiment of the therapy catheter 70 similar to the catheter in FIG. 3 except for the introduction of a dog-bone shaped balloon 72 to retain the agent in a cavity between the balloon 72 and the vessel wall 30 during irradiation. FIG. 6A shows the balloon in its collapsed configuration as the catheter 70 is inserted in the target vessel 30. To remove blood from the treatment site and to deliver the agent to the vessel wall 30, a buffer solution 75 containing the agent is injected into the vessel through a delivery lumen 71 that opens proximal to the balloon 72. The solution fills the vessel downstream of the delivery lumen 71 (FIG. 6B). In FIG. 6C, the balloon 72 is inflated trapping the solution between the inflated ends of the balloon 72. The rest of the solution is flushed downstream. The vessel wall 30 is irradiated by the LEDs 78. Perfusion is maintained through a perfusion lumen 73 that opens both proximal and distal to the balloon 72, and thus provides a bypass fluid conduit.

When a photodynamic agent is in the solution during irradiation, a considerable amount of the irradiation energy may be absorbed by the agent before it reaches the vessel wall. In this case it may be advantages to clear the path of the irradiation light from any remaining agent once the vascular tissue has absorbed a sufficient concentration of the agent for crosslinking. This can be accomplished by deflating the balloon in FIG. 6C, purging the vessel with saline, and re-inflating the balloon. The trapped saline in the dog-bone balloon provides an unobstructed light path for the irradiation. This approach is simple but has the disadvantage that the agent may leach out of the tissue during irradiation.

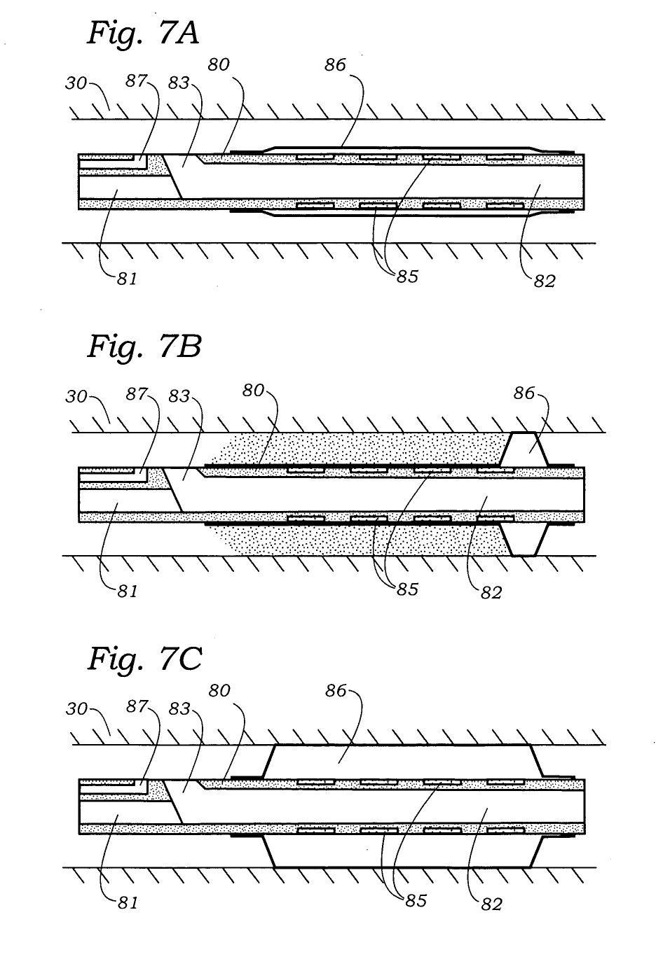

In FIGS. 7A-7C an alternative design of a delivery catheter 80 is shown that retains the agent in the vessel wall. The catheter has a proximal guidewire lumen 81 that is aligned with and communicates with a distal perfusion lumen 82. The inlet 83 into the perfusion lumen 82 is located at the proximal end of the perfusion lumen. The agent is injected through the delivery lumen 87. An array of LEDs 85 is mounted on the distal end of the catheter and covered with an elastomeric balloon 86. The wall thickness of the elastomeric balloon 86 is thinner on its distal end causing the distal portion of the balloon to inflate first, as seen in FIG. 7B, followed by the inflation of the remainder of the balloon as fluid is injected into the balloon 86 (FIG. 7C).

The delivery procedure for the catheter in FIGS. 7A-7C is as follows: First the crosslinking agent is injected through the delivery lumen 87. The balloon 86 is partially inflated with saline as in FIG. 7B to occlude the vessel at the distal end of the catheter and trap the agent in the space between the inlet 83 of the perfusion lumen 82 and the balloon 86. Once the agent has penetrated the vessel wall, the balloon 86 is fully inflated as in FIG. 7C. The agent is pushed out of the vessel lumen creating a clear light path for the array of LEDs 85. The wall of the balloon 86 prevents the agent from leaching out of the vessel wall during subsequent irradiation. During partial or complete balloon inflation, perfusion is maintained through the perfusion lumen 82 that opens both proximal and distal to the balloon 86, and thus provides a bypass fluid conduit.

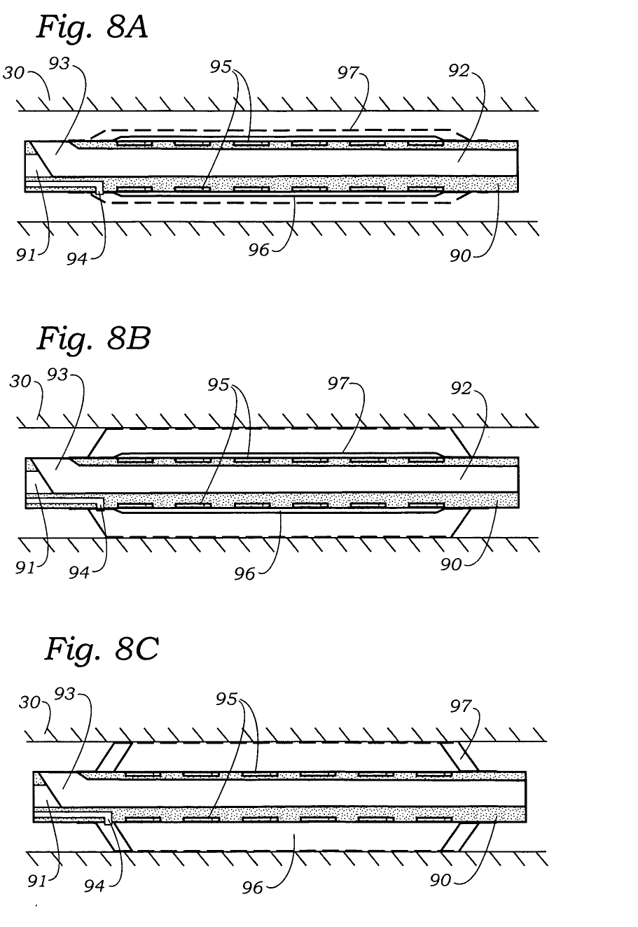

In FIGS. 8A-8C another embodiment of the delivery catheter 90 is shown similar to the catheter in FIG. 7. The catheter has a guidewire lumen 91 connecting to a perfusion lumen 92 with an inlet 93 proximal to an array of LEDs 95. The LEDs 95 are covered with an elastomeric balloon 96. A permeable balloon 97 is mounted over the inner balloon 96 and connected to the delivery lumen 94.

The catheter is placed in the target vessel and the balloon 97 is inflated by injecting the agent into the delivery lumen 94. As shown in FIG. 8B, the balloon 97 displaces the blood in the vessel lumen. The balloon 97 is permeable to the particular agent and thus the agent leached through the balloon 97 into the vessel wall 30. Continuous perfusion is provided by the perfusion lumen 92. Once the agent has penetrated the tissue, the proximal end of the delivery lumen 94 is opened to deflate the outer balloon 97, and the inner balloon 96 is inflated with a clear fluid as seen in FIG. 8C. With the inner balloon 96 inflated, and a clear optical path created, the vessel wall having the agent therein is irradiated.

While the foregoing describes the preferred embodiments of the invention, various alternatives, modifications, and equivalents may be used. Moreover, it will be obvious that certain other modifications may be practiced within the scope of the appended claims.

What is claimed is:

1. A method for crosslinking an extra cellular matrix layer in the vascular system of the body comprising:
providing a vascular catheter;
delivering a crosslinking agent to the extra cellular matrix layer with the vascular catheter; and
irradiating the extra cellular matrix layer and crosslinking agent with light energy emitted from the vascular catheter.

2. A method according to claim 1, wherein the crosslinking agent contains Riboflavin or Riboflavin-5-phosphate.

3. A method according to claim 2, wherein the wavelength of the irradiation energy is between about 200 nm and 500 nm.

4. A method according to claim 3, wherein the wavelength of the irradiation energy is between about 320 nm and 400 nm.

5. A method according to claim 1, wherein the crosslinking agent contains a saccharide or a phosphate derivative thereof.

6. A method according to claim 5, wherein the wavelength of the irradiation energy is between about 150 nm and 400 nm.

7. A method according to claim 6, wherein the crosslinking agent further contains traces of metals.

8. A method according to claim 5, wherein the crosslinking agent further contains a photosensitizer that generates oxygen radicals when irradiated.

9. A method according to claim 5, wherein the crosslinking agent further contains hydrogen peroxide.

10. A method according to claim 8, wherein the crosslinking agent further contains traces of metals.

11. A method according to claim 9, wherein the crosslinking agent further contains traces of metals.

12. A method according to claim 1, wherein the vascular catheter includes one or more light emitting diodes mounted thereon which provide the energy for the step of irradiating.

13. A method for crosslinking an extra cellular matrix layer in the vascular system of the body comprising:
delivering a saccharide to the extra cellular matrix layer; and
irradiating the extra cellular matrix layer and saccharide with light energy to crosslink the extra cellular matrix layer.

14. A method according to claim 13, wherein the wavelength of the light energy is between about 150 and 450 nm.

15. A method according to claim 14, wherein the saccharide further contains traces of metals.

16. A method according to claim 13, wherein the saccharide further contains a photosensitizer that generates oxygen radicals when irradiated.

17. A method according to claim 13, wherein the saccharide further contains traces of metals.

18. A method according to claim 13, wherein the saccharide further contains hydrogen peroxide.

19. A method according to claim 18, wherein the saccharide further contains traces of metals.

20. A method according to claim 13, wherein the saccharide is selected from the group consisting of:
glucose or a phosphate derivative thereof;
ribose or a phosphate derivative thereof; and
fructose or a phosphate derivative thereof.

21. A method according to claim 13, wherein the method includes delivering the saccharide to the extra cellular matrix layer and irradiating the extra cellular matrix layer and saccharide with light energy using a dual-purpose catheter.

22. A method for crosslinking an extra cellular matrix layer in the vascular system of the body comprising:
delivering an agent selected from the group consisting of Riboflavin and Riboflavin-5-phosphate to the extra cellular matrix layer; and
irradiating the extra cellular matrix layer and Riboflavin or Riboflavin-5-phosphate with light energy to crosslink the extra cellular matrix layer.

23. A method according to claim 22, wherein the wavelength of the light energy is between about 200 nm and 500 nm.

24. A method according to claim 23, wherein the wavelength of the light energy is about 220-225 nm, 266 nm, 371 nm, 444 nm, or 475 nm.

25. A method according to claim 22, wherein the method includes delivering the Riboflavin or Riboflavin-5-phosphate to the extra cellular matrix layer and irradiating the extra cellular matrix layer and Riboflavin or Riboflavin-5-phosphate with light energy using a dual-purpose catheter.

26. A method according to claim 22, wherein the agent further contains hydrogen peroxide.

27. A method according to claim 22, wherein the agent further contains traces of metals.

28. A method for crosslinking an extra cellular matrix layer in the vascular system of the body comprising:
delivering a photoxidizer to the extra cellular matrix layer; and
irradiating the extra cellular matrix layer and photooxidizer with light energy to crosslink the extra cellular matrix layer.

29. A vascular catheter for delivering light energy to a blood vessel wall comprising:
a light-emitting diode (LED) on the distal end of the catheter,
a transparent balloon mounted over the LED; and
a lumen opening distal and proximal to the balloon.

30. The catheter of claim 29, wherein the lumen is of sufficient size for blood perfusion therethrough.

31. The catheter of claim 29, further including a guidewire, wherein the lumen is of sufficient size to function as a lumen for passage of the guidewire.

32. The catheter of claim 29, wherein the balloon is mounted eccentrically onto the balloon to create a void between the catheter and the body vessel for perfusion of the vessel distal to the catheter.

33. The catheter of claim 29, wherein the balloon forms a cavity between the outer surface of the balloon and the vessel wall for holding a therapeutic agent.

34. The catheter of claim 33, wherein the balloon has a dog-bone shape.

35. The catheter of claim 29, further comprising:
a photo-sensitive agent contained in the balloon.

36. The catheter of claim 29, wherein there are at least two balloons mounted parallel to each other, and wherein the inflation of the balloons creates a void between the balloons, the catheter, and the vessel wall for perfusion of the vessel distal to the catheter.

37. The catheter of claim 36, wherein there are just two balloons mounted on opposite sides of the catheter and one is larger than the other and covers the LED.

38. The catheter of claim 36, wherein there are four balloons mounted along longitudinal quadrants of the catheter and connected to at least two inflation lumens.

39. The catheter of claim 36, wherein there are four arrays of axially spaced LEDs mounted along the catheter and beneath the respective balloons.

40. The catheter of claim 29, wherein the balloon is permeable.

41. The catheter of claim 29, wherein there is a permeable balloon and a non-permeable balloon, and wherein the non-permeable balloon is contained within the permeable balloon.

42. The catheter of claim 40, wherein the balloon contains a photo-sensitive agent.

43. The catheter of claim 40, wherein the balloon contains a crosslinking agent.
